(19) United States
(12) Patent Application Publication
(10) Pub. No.: US 2021/0282880 A1
(43) Pub. Date: Sep. 16, 2021

(54) PACKAGING CLIP FOR SURGICAL INSTRUMENTS HAVING UNDERCUT GEOMETRY

(71) Applicant: Covidien LP, Mansfield, MA (US)

(72) Inventors: Douglas M. Pattison, East Hartford, CT (US); Oksana Buyda, East Haven, CT (US); Amanda M. Adinolfi, Wallingford, CT (US); Christopher A. Tokarz, Torrington, CT (US)

(21) Appl. No.: 16/815,633

(22) Filed: Mar. 11, 2020

(51) Int. Cl. A61B 50/30; A61M 5/00

(52) U.S. Cl. CPC A61B 50/3001; A61M 5/002

(57) ABSTRACT

A packaging clip includes a first portion detachably securing a cannula assembly thereto, a second portion detachably securing a syringe thereto, and a third portion detachably securing an obturator thereto. The first portion includes an arcuate portion and a plurality of fingers. The arcuate portion secures a sleeve portion of a fluid port of the cannula assembly thereto such that the sleeve portion is disposed orthogonal to a longitudinal axis defined by the packaging clip. The plurality of fingers includes a first finger extending from a first lateral side of the packaging clip and second and third fingers extending from a second lateral side of the packaging clip. The plurality of fingers secures the lateral portion of the fluid port such that the lateral portion extends along the longitudinal axis of the packaging clip. The first portion of the packaging clip is formed by straight-pull molding.

### FIELD

The disclosure relates generally to packaging clips for securing surgical instruments thereto, and more particularly, to a packaging clip having undercut geometry.

### BACKGROUND

Minimally invasive surgical procedures, including endoscopic, laparoscopic and arthroscopic procedures, have been used for introducing surgical instruments inside a patient and for viewing portions of the patient's anatomy. Forming a relatively small diameter, temporary pathway to the surgical site is a key feature of most minimally invasive surgical procedures. The most common method of providing such a pathway is by inserting a trocar assembly through the skin.

Obturators are typically designed with a tip that may be used to form an opening through the abdominal wall. An obturator is inserted into a trocar cannula, and then the combined obturator and trocar cannula are together placed against the skin to be penetrated. In order to penetrate the skin, the distal end of the obturator engages the skin, which may or may not have been previously cut with a scalpel. The obturator is then used to penetrate the skin and access the body cavity. By applying pressure against the proximal end of the obturator, the tip of the obturator is forced though the skin and the underlying tissue layers until the cannula and obturator enter the body cavity. The obturator is then withdrawn. The cannula remains in place within the incision for use during the minimally invasive procedure.

When such surgical instruments are packaged for storage or transport, movement of the surgical instruments within a packaging during storage or transport of the surgical instruments may damage the surgical instrument and/or the packaging material. When the packaging material is damaged, the sterile surgical instruments may be compromised and no longer be suitable for use. Accordingly, there is a need for a packaging clip that effectively and safely secures surgical instruments during storage and transport of the surgical instruments.

### SUMMARY

In accordance with the disclosure, a surgical kit includes a cannula assembly, a syringe, an obturator, and a packaging clip. The cannula assembly has a housing, an elongate shaft defining a lumen and extending from the housing, and a fluid port including a sleeve portion and a lateral portion extending laterally from the sleeve portion and adapted to be coupled to a fluid source. The syringe includes a barrel defining a chamber and having a barrel flange, and a plunger slidably received in the chamber and having a plunger flange. The obturator includes a head portion including latches configured to engage respective notches defined in the housing of the cannula assembly, an elongate member extending from the head portion and dimensioned to be received through the lumen of the elongate shaft of the cannula assembly, and a tip portion coupled to a distal end of the elongate member.

The packaging clip includes first, second, and third portions. The first portion detachably secures the cannula assembly thereto. The first portion includes an arcuate portion and a plurality of fingers. The arcuate portion is configured to secure the sleeve portion of the fluid port of the cannula assembly thereto such that the sleeve portion is disposed orthogonal to a longitudinal axis defined by the packaging clip. The plurality of fingers includes a first finger extending from a first lateral side of the packaging clip and second and third fingers extending from a second lateral side of the packaging clip. The plurality of fingers is configured to secure the lateral portion of the fluid port such that the lateral portion extends along the longitudinal axis of the packaging clip. The second portion detachably secures the syringe thereto. The second portion includes a first holding structure configured to secure the barrel of the syringe thereto such that the syringe is positioned orthogonal to the longitudinal axis. The third portion detachably secures the obturator thereto. The third portion includes a second holding structure configured to secure the elongate member of the obturator such that the elongate member of the obturator is orthogonal to the longitudinal axis. The first portion of the packaging clip is formed by straight-pull molding.

In an aspect, the second and third portions of the packaging clip may be formed by straight-pull molding.

In another aspect, the first, second, and third fingers of the packaging clip may be spaced apart.

In yet another aspect, the second and third fingers of the packaging clip may be disposed on respective sides of the first finger.

In an aspect, the first finger may have a slight bend towards the second lateral side of the packaging clip, and the second and third fingers may have a slight bend towards the first lateral side of the packaging clip.

In another aspect, the first holding structure of the second portion may define an arcuate profile.

In yet another aspect, the first holding structure of the second portion may extend laterally outwards from the second lateral side of the packaging clip.

In still yet another aspect, the plunger flange of the syringe may engage the first lateral side of the packaging clip when the barrel is secured to the second portion of the packaging clip.

In still yet another aspect, a diameter of the first holding structure may be smaller than a diameter of the barrel flange of the syringe.

In an aspect, the housing of the cannula assembly may further include an insufflation connector extending radially outwards from the housing such that the insufflation connector is disposed laterally outwards of the first lateral side of the packaging clip.

In another aspect, the third portion of the packaging clip may include opposing guards configured to engage respective latches of the head portion of the obturator.

In yet another aspect, the second holding structure of the third portion of the packaging clip may be interposed between the opposing guards.

In still yet another aspect, the packaging clip may be monolithically formed.

In still yet another aspect, the packaging clip may be formed of elastomer.

In still yet another aspect, the second portion of the packaging clip may be interposed between the first and third portions.

In accordance with another aspect of the disclosure, a packaging clip for use with surgical instruments includes first, second, and third portions. The first portion detachably secures a cannula assembly thereto. The first portion includes an arcuate portion and a plurality of fingers. The arcuate portion is configured to secure a first part of a fluid port of the cannula assembly thereto such that the first part of the fluid port is disposed orthogonal to a longitudinal axis defined by the packaging clip. The plurality of fingers includes a first finger extending from a first lateral side of the packaging clip and second and third fingers extending from a second lateral side of the packaging clip. The plurality of fingers is configured to secure a second part of the fluid port such that the second part of the fluid port extends along the longitudinal axis of the packaging clip. The second portion detachably secures the syringe thereto. The second portion includes a first holding structure configured to secure a barrel of the syringe thereto such that the syringe is positioned orthogonal to the longitudinal axis. The third portion detachably secures an obturator thereto. The third portion includes a second holding structure configured to secure an elongate member of the obturator such that the elongate member of the obturator is orthogonal to the longitudinal axis. The packaging clip is formed by straight-pull molding.

In an aspect, the first portion of the packaging clip may be configured to eliminate six degrees of freedom of the cannula assembly secured thereto.

In another aspect, the third portion of the packaging clip includes opposing guards configured to engage respective latches of a head portion of the obturator.

In yet another aspect, the second holding structure of the third portion of the packaging clip may be interposed between the opposing guards.

In still yet another aspect, the second holding structure of the third portion may have a C-shape extending between the first and second lateral sides of the packaging clip.

### BRIEF DESCRIPTION OF DRAWINGS

A packaging clip for use with surgical instruments is disclosed herein with reference to the drawings, wherein:

FIG. 1 is a perspective view of a surgical kit including a cannula assembly, a syringe, an obturator, and a packaging clip in accordance with the disclosure;

FIG. 2 is a perspective view of the packaging clip of FIG. 1;

FIG. 3 is a front view of the surgical kit of FIG. 1;

FIG. 4 is an enlarged front view of the indicated area of detail of FIG. 3;

FIG. 5 is a top view of the packaging clip of FIG. 2;

FIG. 6 is a front view of the packaging clip of FIG. 2;

FIG. 7 is a rear view of the packaging clip of FIG. 2;

FIG. 8 is a bottom view of the packaging clip of FIG. 2;

FIG. 9 is a perspective view of the packaging clip of FIG. 2, illustrating direction of straight-pull molding; and

FIG. 10 is a front view of the packaging clip of FIG. 9, illustrating direction of straight-pull molding.

### DETAILED DESCRIPTION

A packaging clip for use with surgical instruments is described in detail with reference to the drawings, wherein like reference numerals designate corresponding elements in each of the several views. As used herein, the term “distal” refers to that portion of the instrument, or component thereof which is farther from the user while the term “proximal” refers to that portion of the instrument or component thereof which is closer to the user. In addition, the terms parallel and perpendicular are understood to include relative configurations that are substantially parallel and substantially perpendicular up to about + or −10 degrees from true parallel and true perpendicular. Further, to the extent consistent, any or all of the aspects detailed herein may be used in conjunction with any or all of the other aspects detailed herein.

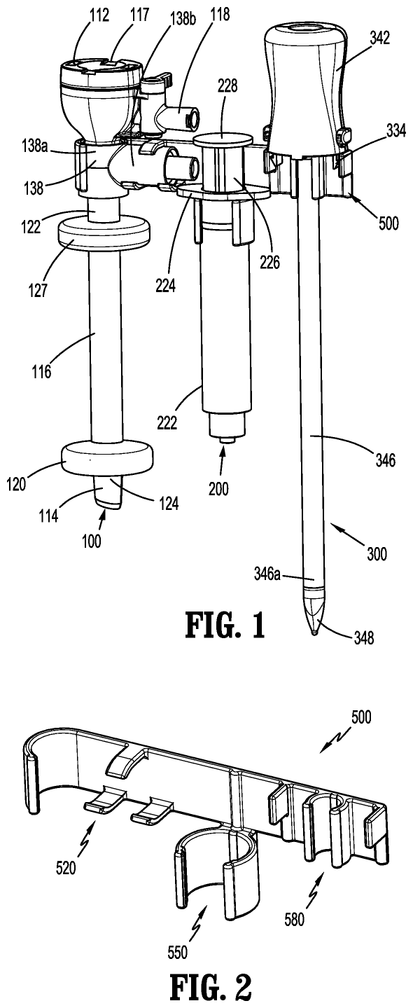

With reference to FIGS. 1 and 2, a packaging clip for use with surgical instruments, in the form of a cannula assembly 100, a syringe 200, or an obturator 300 is shown generally as 500. The obturator 300 may be an optical obturator. The packaging clip 500 may be integrally formed or, alternatively, monolithically formed. The packaging clip 500 is configured to secure the cannula assembly 100, the syringe 200, and the obturator 300 thereto. The packaging clip 500 includes a first portion 520 configured to detachably secure the cannula assembly 100 thereto, a second portion 550 configured to detachably secure the syringe 200 thereto, and a third portion 580 configured to detachably secure the obturator 300 thereto. The second portion 550 may be interposed between the first and third portions 520, 580. The packaging clip 500 is molded from a compliant material such as, e.g., elastomer or plastic. The material and the geometry such as, e.g., shape and thickness, of the packaging clip 500 facilitate attachment and detachment of the cannula assembly 100, the syringe 200, or the obturator 300 to and from the packaging clip 500.

The packaging clip 500 includes undercut geometry that is formed by straight-pull molding. An undercut is a feature in the part design that would prevent the part from releasing out of the mold (part of the mold cavity is undercut in such a way that it grips the molded part). In straight-pull molding, first and second sides of the mold open and the completed part can be removed by pulling it straight out of either side. In contrast, side-pull molding enables undercut geometry that could not otherwise be made in a straight-pull mold. To release a part that includes undercuts, the mold surfaces that create the undercut geometry in the part is pulled out of the part before it is ejected; otherwise the part will be stuck in the mold. Accordingly, undercuts increase the complexity of the molds by introducing side-pulls which limit the number of cavities in the mold's frame, which, reduces the number of parts manufactured. As such, the packaging clip 500 formed by straight-pull molding is advantageous over the side-pull molding. The packaging clip 500 provides multiple holding structures to fully constrain surgical instruments thereto by providing undercut configurations that are formed by straight-pull molding. In this manner, the packaging clip 500 formed by straight-pull molding reduces mold cycle times, complexity of molds, and need for mold maintenance, but increases number of cavities allowed in a mold.

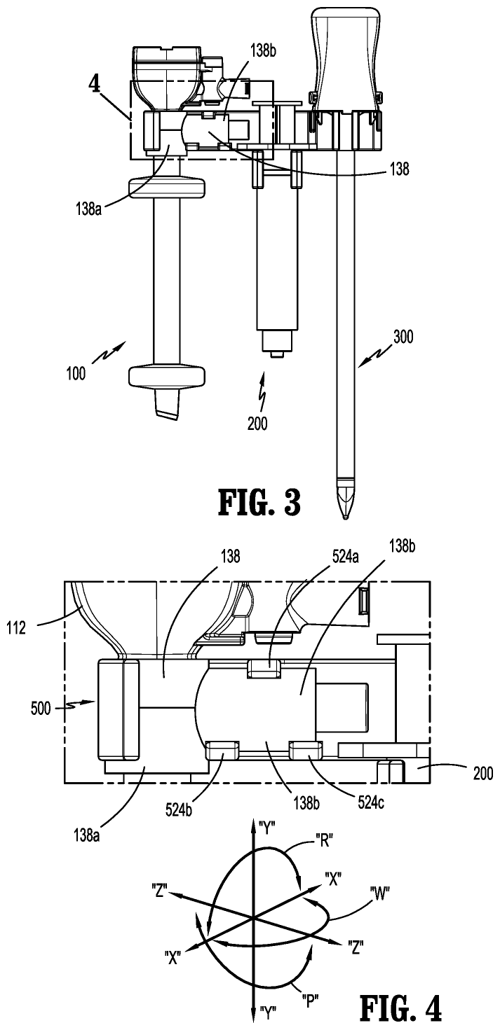

With reference to FIGS. 1-3, the first portion 520 is configured to releasably secure the cannula assembly 100 thereto. The cannula assembly 100 is configured to permit access to an insufflated abdominal cavity during a laparoscopic procedure to permit the introduction of a surgical object for performing various surgical tasks on internal organs within the cavity such as, e.g., removal of surgical specimen. The surgical object may be a surgical instrument such as laparoscopic or endoscopic clip appliers, graspers, dissectors, retractors, staplers, laser probes, photographic devices, tubes, endoscopes and laparoscopes, electro-surgical devices and the like. The cannula assembly 100 generally includes a cannula housing 112, a cannula member 114 extending from the cannula housing 112 and an outer sleeve 116 coaxially mounted over the cannula member 114, an expandable balloon 120, and a locking collar 127 positioned about the proximal end 122 of the cannula assembly 100 and advanced to engage the exterior surface of tissue (e.g., the abdominal wall). The locking collar 127 in combination with the expandable balloon 120 minimizes movement of the cannula member 114 in both withdrawal and insertion directions and also assists in maintaining a seal about the passage in the abdominal wall.

The cannula housing 112 is dimensioned for engagement by the clinician and may include one or more internal seals (not shown) adapted to establish a seal about a surgical object introduced therethrough. The cannula housing 112 also may include an insufflation connector 118 (e.g., a luer connector) for connecting to a source of insufflation fluids (not shown) for delivery within, e.g., the abdominal cavity. A longitudinal lumen defined by the cannula member 114 is also in fluid communication with the insufflation connector 118 to convey insufflation fluids into the abdominal cavity to establish and/or maintain the pneumoperitoneum. The expandable balloon 120 is coupled to the outer sleeve 116. The cannula member 114 further includes a fluid port 138 positioned adjacent the cannula housing 112. The fluid port 138 is configured for coupling to a source of inflation fluids to inflate the expandable balloon 120. The fluid port 138 includes a sleeve portion 138a disposed about the cannula member 114, and a lateral portion 138b extending laterally from the sleeve portion 138a and configured to be coupled to the source of the inflation fluid. The outer sleeve 116 is coaxially mounted about the cannula member 114 and extends from a position within the fluid port 138 to a position adjacent the distal end 124 of the cannula member 114. The outer sleeve 116 may be secured within the fluid port 138 and to the cannula member 114 through a friction or interference fit or with the use of adhesives, cements or the like. The expandable balloon 120 is coupled to the outer sleeve 116 and is coaxially mounted about the distal end 124 of the cannula member 114. The expandable balloon 120 expands radially outwardly upon passage of inflation fluids through the fluid port 138. In an aspect, the outer sleeve 116 may be mounted over the fluid port 138.

The syringe 200 is configured to be secured to the second portion 550 of the packaging clip 500. The syringe 200 generally includes a barrel 222 having a barrel flange 224, and a plunger 226 slidably received in the barrel 222 and having a plunger flange 228. The obturator 300 is configured to be secured to the third portion 580 of the packaging clip 500. The obturator 300 generally includes a head portion 342 having latches 334 configured to engage respective notches 117 defined in the cannula housing 112 of the cannula assembly 100 to enhance securement therewith, an elongate shaft 346 extending from the head portion 342, and an optical penetrating tip 348 coupled to a distal end 346a of the elongate shaft 346.

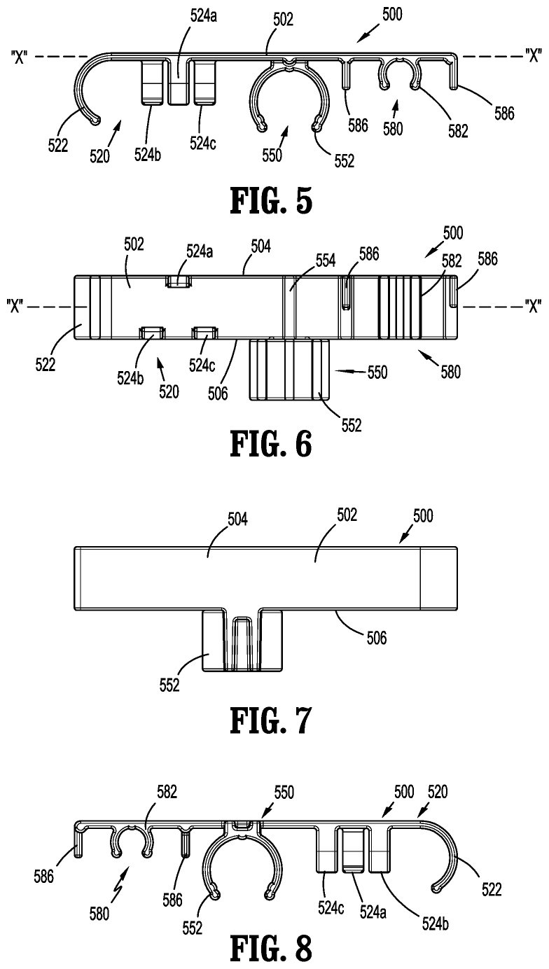

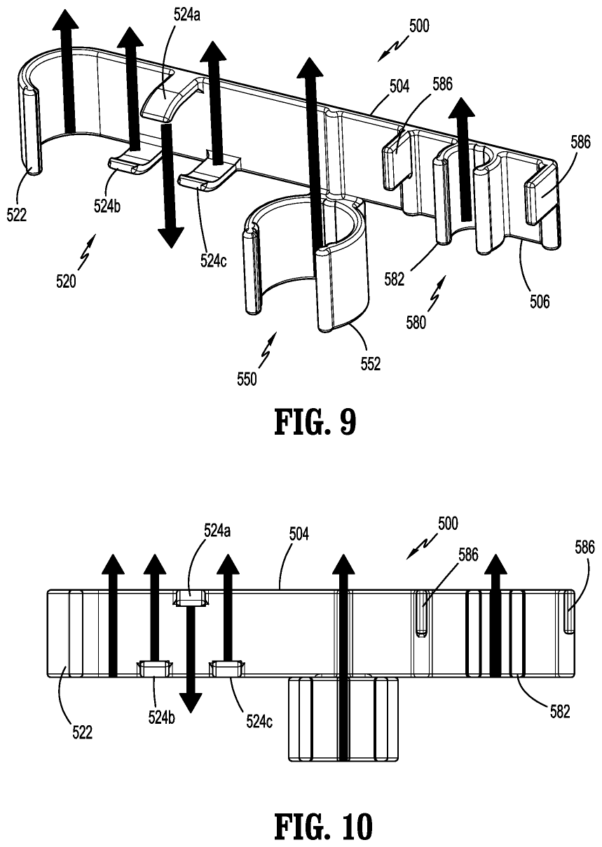

With particular reference to FIGS. 4-8, the first portion 520 of the packaging clip 500 is configured to secure the cannula assembly 100 thereto. In particular, the first portion 520 includes an arcuate portion 522 that bends outwardly (e.g., in the direction of an axis “Z-Z”) such that the arcuate portion 522 is offset from a longitudinal axis “X-X” defined by the packaging clip 500. The arcuate portion 522 conforms to a curvature profile of a sleeve portion 138a (FIG. 1) of the fluid port 138 such that the arcuate portion 522 wraps around a portion of the fluid port 138. In addition, the first portion 520 further includes first, second, and third fingers 524a, 524b, 524c extending from a body 502 of the packaging clip 500. In particular, a first finger 524a may extend from a first lateral side 504 of the body 502 and second and third fingers 524b, 524c may extend from a second lateral side 506 of the body 502 (FIG. 6). The first finger 524a is offset from the second and third fingers 524b, 524c such that the first finger 524a does not overlap with the second and third fingers 524b, 524c. The first, second, and third fingers 524a, 524b, 524c may also be spaced apart along the length of the packaging clip 500 (FIG. 5). In addition, the first finger 524a further engages the sleeve portion 138a of the fluid port 138 when the sleeve portion 138a is secured with the arcuate portion 522 such that the arcuate portion 522 and first finger 124a inhibit displacement of the fluid port 138 along the longitudinal axis “X-X.” In addition, each finger 524a, 524b, 524c provides a slight bend inward. Such bend may conform to the curvature of the lateral portion 138b of the fluid port 138. The fingers 524a, 524b, 524c as a whole secure the lateral portion 138b of the fluid port 138 between the first and second lateral sides 504, 506 (FIG. 6) of the body 502. Under such a configuration, when the cannula assembly 100 engages the first portion 520 of the packaging clip 500, the first portion 520 removes 6 degrees of freedom in the axes “X-X,” “Y-Y,” “Z-Z,” and arrows R, W, P (FIG. 4) and secures the cannula assembly 100 thereto. As can be appreciated with reference to FIGS. 9 and 10, the arcuate portion 522 and the first, second, and third fingers 524a, 524b, 524c that have an undercut geometry may be obtained through straight-pull molding. It is contemplated that a protrusion may extend laterally outwards from the lateral side 504 of the body 502 in order to protect a pouch from rotation of the insufflation connector 118 of the cannula housing 112 in the direction of arrow “W”. With continued reference to FIGS. 5-8, the second portion 550 of the packaging clip 500 is configured to secure the syringe 200 thereto. The second portion 550 includes a first holding structure 552 extending laterally outwards from the second lateral side 506 of the body 502. The first holding structure 552 has an arcuate configuration that is, e.g., C-shaped. The barrel 222 of the syringe 200 is secured with the first holding structure 552, and the barrel flange 224 is disposed on the first holding structure 552 and engages the body 502. In addition, the plunger flange 228 engages the first lateral side 504 of the body 502 when the plunger 226 is received in the barrel 222 of the syringe 200. The second portion 550 of the packaging clip 500 may include a rib 554 extending between the first and second lateral sides 504, 506 to enhance structural integrity of the packaging clip 500. Under such a configuration, the syringe 200 is secured to the packaging clip 500 such that the syringe 200 is orthogonal to the longitudinal axis “X-X.” The first holding structure 552 is formed by straight-pull molding as shown in FIGS. 9 and 10.

With continued reference to FIGS. 5-8, the third portion 580 of the packaging clip 500 includes a second holding structure 582 disposed on the body 502 and extending between the first and second lateral sides 504, 506. The second holding structure 582 has an arcuate configuration that is, e.g., C-shaped, and opposing guards 586 spaced apart from respective sides of the second holding structure 582. Each guard 586 is orthogonal to the longitudinal axis “X-X” of the packaging clip 500 and is disposed between the first and second lateral sides 504, 506. In particular, each guard 586 extends partially or completely between the first and second lateral sides 504, 506. Under such a configuration, the elongate shaft 346 (FIG. 1) of the obturator 300 is secured with the second holding structure 582 such that the elongate shaft 346 is orthogonal to the longitudinal axis “X-X.” In addition, the head portion 342 of the obturator 300 rests on the second holding structure 582 and the latches 334 extending from the head portion 342 engage the respective guards 586 of the third portion 580 of the packaging clip 500. In this manner, the obturator 300 is secured to the body 502. Such a configuration may be formed by straight-pull molding as shown in FIGS. 9 and 10.

The packaging clip 500 includes undercut geometry to securely attach the cannula assembly 100, the syringe 200, and the obturator 300 thereto. The packaging clip 500 may be formed by straight-pull molding. In this manner, the packaging clip 500 reduces mold tooling costs, mold cycle times, complexity of molds and mold maintenance. It is envisioned that the packaging clip 500 may be configured to support the cannula assembly 100, the syringe 200, and the obturator 300 in, e.g., a different order or orientation. For example, the obturator 300 may be interposed between the cannula 100 assembly and the syringe 200.

Persons skilled in the art will understand that the devices and methods specifically described herein and illustrated in the accompanying drawings are non-limiting. It is envisioned that the elements and features may be combined with the elements and features of another without departing from the scope of the disclosure. As well, one skilled in the art will appreciate further features and advantages of the disclosure.

What is claimed is:

1. A surgical kit comprising:
a cannula assembly having a housing, an elongate shaft defining a lumen and extending from the housing, and a fluid port including a sleeve portion and a lateral portion extending laterally from the sleeve portion and adapted to be coupled to a fluid source;
a syringe including a barrel defining a chamber and including a barrel flange, and a plunger slidably received in the chamber and having a plunger flange;
an obturator including a head portion including latches configured to engage respective notches defined in the housing of the cannula assembly, an elongate member extending from the head portion and dimensioned to be received through the lumen of the elongate shaft of the cannula assembly, and a tip portion coupled to a distal end of the elongate member; and
a packaging clip including:
a first portion detachably securing the cannula assembly thereto, the first portion including:
an arcuate portion configured to secure the sleeve portion of the fluid port of the cannula assembly thereto such that the sleeve portion is disposed orthogonal to a longitudinal axis defined by the packaging clip; and
a plurality of fingers including a first finger extending from a first lateral side of the packaging clip and second and third fingers extending from a second lateral side of the packaging clip, the plurality of fingers configured to secure the lateral portion of the fluid port such that the lateral portion extends along the longitudinal axis of the packaging clip;
a second portion detachably securing the syringe thereto, the second portion including a first holding structure configured to secure the barrel of the syringe thereto such that the syringe is positioned orthogonal to the longitudinal axis; and
a third portion detachably securing the obturator thereto, the third portion including a second holding structure configured to secure the elongate member of the obturator such that the elongate member of the obturator is orthogonal to the longitudinal axis,
wherein the first portion of the packaging clip is formed by straight-pull molding.

2. The surgical kit according to claim 1, wherein the second and third portions of the packaging clip are formed by straight-pull molding.

3. The surgical kit according to claim 1, wherein the first, second, and third fingers of the packaging clip are spaced apart.

4. The surgical kit according to claim 1, wherein the second and third fingers of the packaging clip are disposed on respective sides of the first finger.

5. The surgical kit according to claim 1, wherein the first finger has a slight bend towards the second lateral side of the packaging clip, and the second and third fingers have a slight bend towards the first lateral side of the packaging clip.

6. The surgical kit according to claim 1, wherein the first holding structure of the second portion defines an arcuate profile.

7. The surgical kit according to claim 1, wherein the first holding structure of the second portion extends laterally outwards from the second lateral side of the packaging clip.

8. The surgical kit according to claim 1, wherein the plunger flange of the syringe engages the first lateral side of the packaging clip when the barrel is secured to the second portion of the packaging clip.

9. The surgical kit according to claim 1, wherein a diameter of the first holding structure is smaller than a diameter of the barrel flange of the syringe.

10. The surgical kit according to claim 1, wherein the housing of the cannula assembly further includes an insufflation connector extending radially outwards from the housing such that the insufflation connector is disposed laterally outwards of the first lateral side of the packaging clip.

11. The surgical kit according to claim 1, wherein the third portion of the packaging clip includes opposing guards configured to engage respective latches of the head portion of the obturator.

12. The surgical kit according to claim 1, wherein the second holding structure of the third portion of the packaging clip is interposed between the opposing guards.

13. The surgical kit according to claim 1, wherein the packaging clip is monolithically formed.

14. The surgical kit according to claim 1, wherein the packaging clip is formed of elastomer.

15. The surgical kit according to claim 1, wherein the second portion of the packaging clip is interposed between the first and third portions.

16. A packaging clip for use with surgical instruments comprising:
a first portion detachably securing a cannula assembly thereto, the first portion including:
an arcuate portion configured to secure a first part of a fluid port of the cannula assembly thereto such that the first part of the fluid port is disposed orthogonal to a longitudinal axis defined by the packaging clip; and
a plurality of fingers including a first finger extending from a first lateral side of the packaging clip and second and third fingers extending from a second lateral side of the packaging clip, the plurality of fingers configured to secure a second part of the fluid port such that the second part of the fluid port extends along the longitudinal axis of the packaging clip;
a second portion detachably securing the syringe thereto, the second portion including a first holding structure configured to secure a barrel of the syringe thereto such that the syringe is positioned orthogonal to the longitudinal axis; and
a third portion detachably securing an obturator thereto, the third portion including a second holding structure configured to secure an elongate member of the obturator such that the elongate member of the obturator is orthogonal to the longitudinal axis,
wherein the packaging clip is formed by straight-pull molding.

17. The packaging clip according to claim 16, wherein the first portion of the packaging clip is configured to eliminate six degrees of freedom of the cannula assembly secured thereto.

18. The packaging clip according to claim 16, wherein the third portion of the packaging clip includes opposing guards configured to engage respective latches of a head portion of the obturator.

19. The packaging clip according to claim 18, wherein the second holding structure of the third portion of the packaging clip is interposed between the opposing guards.

20. The packaging clip according to claim 16, wherein the second holding structure of the third portion has a C-shape extending between the first and second lateral sides of the packaging clip.
